(19) United States
(12) Patent Application Publication
(10) Pub. No.: US 2010/0042119 A1
(43) Pub. Date: Feb. 18, 2010

(54) SYSTEMS AND METHODS FOR SECURING SUBCUTANEOUS IMPLANTED DEVICES

(75) Inventors: William J. Simms, Lousiville, CO (US); Scott Allan Miller, III, Golden, CO (US); Nicholas Pergola, Arvada, CO (US); Brian M. Conn, Broomfield, CO (US); James R. Easter, Lyons, CO (US)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/538,760

(22) Filed: Aug. 10, 2009

Related U.S. Application Data
(60) Provisional application No. 61/087,503, filed on Aug. 8, 2008.

(51) Int. Cl. A61B 17/00

(52) U.S. Cl. 606/151

(57) ABSTRACT

A system for reducing subcutaneous migration of an implantable device or housing relative to surrounding soft tissue. For instance, the implantable housing may support a microphone diaphragm. The system includes at least one securement member having at least one aperture extending therethrough that may selectively receive one of a soft tissue securement device (e.g., soft tissue suture) and soft tissue growth therethrough. The securement member is at least one of interconnected to and disposable over at least a portion of the housing and at least one of extends away from and is selectively extendable away from a periphery of the housing. In one arrangement, at least one mesh member may be optionally included with the system that may allow for tissue growth to enhance securement of the implanted device relative to the soft tissue.

### RELATED APPLICATIONS

This application claims priority to U.S. Provisional Application Ser. No. 61/087,503 filed Aug. 8, 2008, entitled “SYSTEM AND METHODS FOR SECURING SUBCUTANEOUS IMPLANTED DEVICES”, the entirety of which is hereby incorporated by reference.

### FIELD OF THE INVENTION

The present invention relates to implanted devices, e.g., as employed in hearing aid instruments, and more particularly, to implanted devices that are resistant to subcutaneous migration due to, for example, external forces.

### BACKGROUND

In the class of hearing aids generally referred to as implantable hearing instruments, some or all of various hearing augmentation componentry is positioned subcutaneously on, within or proximate to a patient's skull, typically at locations proximate the mastoid process. In a fully implantable hearing instrument, typically all of the components, e.g., the microphone, signal processor, and auditory stimulator, are located subcutaneously. In such an arrangement, an implantable auditory stimulator device is utilized to stimulate a component of the patient's auditory system (e.g., tympanic membrane, ossicles and/or cochlea).

By way of example, one type of implantable transducer includes an electromechanical transducer having a magnetic coil that drives a vibratory actuator. The actuator is positioned to interface with and stimulate the ossicular chain of the patient via physical engagement. (See e.g., U.S. Pat. No. 5,702,342). In this regard, one or more bones of the ossicular chain are made to mechanically vibrate causing stimulation of the cochlea through its natural input, the so-called oval window.

As may be appreciated, hearing instruments that utilize an implanted microphone require that the microphone be positioned at a location that facilitates the transcutaneous receipt of ambient acoustic signals. For such purposes, implantable microphones have heretofore been affixed to the skulls of a patient at a location rearward and upward of the patient's ear (e.g., in the mastoid region). Other systems have identified it as being desirable to form a soft tissue mounting where the microphone is removed from the surface of the skull to reduce the receipt and amplification of skull borne vibrations by the implanted microphone.

### SUMMARY OF THE INVENTION

The inventors of the systems and methods (i.e., utilities) provided herein have recognized that, while the removal of certain components of implanted devices from the surface of a patient's bone may provide a number of benefits such as the attenuation of some forms of biological noise, such soft tissue mounting may raise additional issues. Specifically, while it may be possible to move one or more components of an implantable device to a soft tissue location to eliminate the need of, for example, forming a bone bed for that component, such soft tissue mounted implantable components can in some instances migrate subcutaneously. That is, as opposed to components that are securely affixed to an underlying bone, soft tissue mounted components may have limited subcutaneous movement. This may be especially evident during the healing process immediately after implantation of the component. Furthermore, a portion of the population that utilizes implantable devices has a tendency to manually manipulate these devices transcutaneously. That is, a number of implant wearers are considered “twiddlers” who have a tendency to consciously or subconsciously feel and/or apply forces to subcutaneously located implantable devices. Accordingly, when such devices are mounted in soft tissue, such twiddling may result in damage to the device and/or to tissue surrounding the implantable device. Accordingly, utilities are provided herein that allow for improved interconnection between an implantable component and soft tissue. Stated otherwise, such utilities aid in the reduction of migration of subcutaneously located components and/or reduce the stresses that may be applied to such components.

According to a first aspect, a system is provided that allows for increasing the distance between securing points on an implanted device to allow for attaching the implanted device over a greater surface area. For instance, the implantable device may include a housing that houses one or more components of an implantable system and may be subcutaneously secured to soft tissue. In one arrangement, the housing may support a microphone diaphragm. The system includes at least one securement member having at least one aperture extending therethrough that may selectively receive one of a soft tissue securement device (e.g., soft tissue suture) and soft tissue growth therethrough. The securement member is at least one of interconnected to and disposable over at least a portion of the housing and at least one of extends away from and is selectively extendable away from a periphery of the housing.

In one arrangement, the at least one securement member may be in the form of a leg, wing or arm that is interconnected to and extends outwardly from a portion of the housing (e.g., periphery) and includes at least a first aperture. As previously discussed, this aperture may be utilized to secure (e.g., suture) the securement member to soft tissue. The securement member may be appropriately connected to the housing or may be integrally formed therewith. As another example, the at least one securement member may be in the form of a loop or aperture that allows for securing the housing to underlying tissue.

In a further arrangement, one or more of the securement members may be deformable. As such, the securement member may initially be disposed adjacent to a surface of the housing and the housing may be implanted without extending the securement member if so desired. Alternatively, the securement member may be displaced/extended from the surface of the housing. In this regard, the securement member may have one or more flexible portions that allow for bending of the securement member to a desired shape or orientation. In a further arrangement, such securement members may include one or more apertures that allow for receipt of a suture and/or bone screw. Thus, the outwardly extending securement members may be utilized to affix the housing to soft tissue and/or underlying bone.

In one embodiment, the housing may have a plurality of securement members extending outwardly therefrom. In a further arrangement, securement members may extend radially outward from a center point of the housing. Typically, a proximal end of each securement member may be affixed to the housing. For instance, the plurality of securement members may extend away from the housing in a corresponding plurality of different directions, each including an aperture therethrough adapted for selective receipt of a soft tissue securement device therethrough. Different ones of a plurality of soft tissue securement devices (e.g., tissue sutures) may be selectively receivable through different ones of the apertures of the plurality of securement members and soft tissue. In some scenarios, at least two securement members of the plurality of securement members may extend along an axis that intersects the center of gravity of the housing. Such an arrangement may advantageously reduce movement of the system or assembly relative to overlying tissue by allowing the housing to move with surrounding soft tissue.

In other arrangements of the present aspect, one or more mesh members (e.g. permeable mesh fabric or other types of material) may be optionally included within the system. The inventors have discovered that by strategically locating one or more mesh members with various aspects of the system, soft tissue may ingress or otherwise grow into various portions of the mesh members (e.g., through apertures) to increase or enhance securement of the housing to soft tissue. The at least one securement member may be in the form of a mesh member that is selectively positionable over a portion of the housing and/or an implantable component (e.g., an “implantable device”).

For instance, the securement member may encapsulate or at least cover at least a portion of the implantable device such that tissue may ingress about the housing and thereby isolate the same. A first layer of mesh material may be disposed on a first side of the implantable device and a second layer of mesh material may be disposed on a second side of the implantable device. In one variation, the first and second mesh layers may be interconnected around at least a portion of their periphery. In this regard, the mesh layer may form a sock, sleeve, pocket or other partially closed configuration that allows for receiving the implantable device between opposing mesh layers. Once the mesh is positioned around a portion or the entirety of the implantable device, the mesh material and implantable device may be positioned subcutaneously. The mesh material allows for tissue ingress during the healing process which may make a secure attachment between the mesh material and the tissue. To enhance securement of the implantable device to surrounding soft tissue, one or more soft tissue securement devices (e.g., sutures) may be received through one or more apertures of the mesh member and the surrounding soft tissue. In another arrangement, the mesh material may be appropriately disposed about (e.g., covered, encapsulated) cabling (e.g., a signal wire) interconnecting one or more housings of an implantable device.

In another arrangement, the at least one securement member may be in the form of a leg, loop, arm or wing, and at least one of the above-mentioned mesh members may be appropriately selectively located thereabout. For instance, the mesh member may be laid over one portion of the securement member before the housing is subcutaneously implanted within a patient. Thus, after the housing is implanted, tissue ingress through the mesh member and/or aperture of the securement member during the healing process may securely attach the housing to the surrounding soft tissue. In other embodiments, the mesh member may be in the form of a pocket such that one or more securement members may be inserted into the pocket before implanting the housing within the patient. One or more soft tissue securement devices (e.g., sutures) may be received through one or more apertures of the mesh member and securement member along with the surrounding soft tissue to enhance securement between the housing and the surrounding soft tissue.

As an additional example, the distal end of each arm may be appropriately covered, or encapsulated, with a mesh member. Such mesh member may be biocompatible and allow for tissue ingress during the healing process. Accordingly, a utility may allow for suturing the distal ends of the outwardly extending arm(s) to patient tissue to initially secure the implantable device to soft tissue. Once initially secured, the healing process may begin and tissue may ingress into the mesh material attached to the distal ends of the arm(s). Accordingly, after the tissue ingresses into the mesh material, the securement of the implantable device to the surrounding tissue may be enhanced.

In further scenarios, the housing and securement member(s) may be subcutaneously implanted, and then one or more mesh members may be laid over or otherwise appropriately located about one or more securement members to allow for soft tissue growth through apertures thereof. For instance, after the housing is implanted and one or more securement members are appropriately secured (e.g., via suturing) to the soft tissue, one or more mesh members may be appropriately located about each securement member and its respective one or more soft tissue securement devices (e.g., sutures) to enhance interconnection between the housing and the surrounding soft tissue. In any of the above-noted aspects, the sizing of the housing may be designed to minimize subcutaneous movement. For instance, the aspect ratio of the housing may be increased such that its width is significantly greater than its height. In such an arrangement, any protuberance of the housing through the skin may be reduced, which may reduce the tendency for a user to touch the device. Further, the high aspect ratio may reduce the ability of the device to turn and/or roll. It will be further appreciated that aspect ratios along first and second axes of the housing may be different such that after tissue is healed around the device, rotation about an axis normal to the device may be limited.

According to another aspect, a strain relief element may be provided for a cable (e.g., signal wire) that interconnects first and second implanted components or housings. The strain relief element may be elastic such that it allows for deflection upon a tensile force being applied to the element ends. The relief element may further include a recess channel for receiving the signal wire that may extend between implanted components. When disposed within the recess, the signal wire may be disposed in a curved or jogged (e.g., S-shaped) configuration such that upon applying a force to either end of the signal wire, the strain relief element may expand and thereby permit signal wire expansion between implanted components. That is, the strain relief element may form a relief bend. In one arrangement, the recess of the strain relief element may form a snap fit arrangement for receiving the signal wire.

In another arrangement, the strain relief element may be fixedly interconnected to the signal wire. For instance, the strain relief element may include an elastic block formed over at least a portion of the signal wire. In this regard, the signal wire may be routed through a resilient block in a manner that provides expansion and contraction capabilities for the signal wire. In a further arrangement, one or more elastic anchors may be interconnected to the signal wire. In such an arrangement, first and second elastic anchors may be affixed to underlying tissue (e.g., bone) to provide a relief bend in the signal wire.

Some embodiments of the present invention provide various methodologies associated with one or more implantable housings or components, and in one characterization, a method for use with an implantable housing is provided. The method broadly includes providing at least one of any of the above described securement members, positioning an implantable housing at a subcutaneous location such that the housing is supported by soft tissue and is spaced from a surface of the skull of a patient, and utilizing at least one aperture of the at least one securement member to secure the implantable housing to the soft tissue. The utilization of at least one securement member having an aperture therethrough allows a technician to more securely and effectively subcutaneousloy mount an implantable housing or component to the soft tissue of a patient by reducing subcutaneous migration of the housing.

In one arrangement, the utilizing step may include appropriately disposing a soft tissue securement device through at least one securement member interconnected to a portion of the implantable housing. For instance, the disposing step may include extending a tissue suture through the at least one aperture of the at least one securement member in addition to soft tissue. Before the disposing step, the at least one securement member may be deformed away from the implantable housing. As previously discussed, such a step may be advantageous in appropriately positioning the securement member relative to a desired mounting location (e.g., soft tissue, bone).

Either before or after the soft tissue securement device is appropriately disposed or extended through the securement member, a mesh member may be located over the at least one securement member whereby one or more apertures of the securement and/or mesh members are sized for growth of soft tissue therethrough. In one arrangement, a tissue suture may be extending through the aperture of the securement member and one or more apertures of the mesh member to reinforce the interconnection between the housing and the surrounding soft tissue. In some scenarios, the step of locating the mesh member over the securement member may include inserting the at least one securement member into a pocket formed in the mesh member. Other scenarios contemplate that two or more pieces of mesh material could sandwich one or more securement members. Even further arrangements contemplate that one or more securement members may be appropriately covered with one or more mesh members without securing (e.g., suturing) the securement members to the surrounding soft tissue.

In one setup, the providing step may include providing a plurality of securement members spaced about and interconnected to a periphery of the implantable housing. For instance, the plurality of securement members may extend away from the implantable housing in a corresponding plurality of different directions, and each of the plurality of securement members may include an aperture therethrough that is adapted for selective receipt of a soft tissue securement device therethrough. Here, the disposing step may include extending different ones of a plurality of tissue securement devices (e.g., tissue sutures) through different ones of the apertures of the plurality of securement members and soft tissue. It will be appreciated the one or more mesh members may be appropriately associated with one or more of the securement members as previously discussed or in other manners. In another arrangement, one or more mesh members may be arranged to appropriately encapsulate or at least cover both the housing and a number of securement members.

In another setup, at least one securement member in the form of a mesh member may be provided, and the mesh member may be appropriately disposed over the housing. For instance, the housing may be covered with the mesh member by way of inserting the housing into a pocket formed in the mesh member. In this scenario, it is contemplated that securement members that are interconnected to a portion of the housing (e.g., wings or arms) may or may not be utilized in conjuction with the mesh member to effectively interconnect the housing to the surrounding soft tissue.

In a further arrangement, the method may include routing a signal wire subcutaneously between the implantable housing and another implantable housing that is mounted relative to the skull of the patient. For instance, the signal wire may interconnect a microphone assembly to a signal receiver or other implantable component. As it may be desirable to limit migration of such a signal wire, the method may further include covering the signal wire with any appropriate migration limiting member (e.g., mesh member). For instance, a mesh member may be laid over, or encase, the signal wire to limit movement of the signal wire during any attempted twiddling by the patient or else during movement of other implantable components and housings. In other arrangements, the method may include locating a strain relief member about a length of the signal wire to prevent or otherwise reduce the effects on the signal wire from twiddling with or other movement of the signal wire. For instance, a bend may be formed along the length of the signal wire (e.g., S-shape) to allow for lengthening of the wire and accommodate, for instance, turning of the patient's head.

### BRIEF DESCRIPTION OF THE DRAWINGS

FIG. 1 illustrates a fully implantable hearing instrument.

FIG. 2 illustrates one embodiment of a soft tissue mount of a microphone.

FIGS. 3A-3D illustrate various embodiments of suture loop connectors as applied to an implantable microphone.

FIGS. 4A and 4B illustrate use of a mesh material to limit movement of an implantable microphone.

FIGS. 5A and 5B illustrate an implantable microphone having an increased aspect ratio.

FIGS. 6A-6D variously illustrate an implantable microphone that may be utilized for both soft tissue mounting as well as mounting to cortical bone.

FIGS. 7A-7C illustrate use of a mesh for limiting movement of an implantable signal wire.

FIGS. 8A and 8B illustrate use of a snap-on strain relief element for an implantable cable.

FIG. 9 illustrates use of a resilient S bend strain relief device.

FIG. 10 illustrates a strain relief anchor built into an implantable signal wire.

### DETAILED DESCRIPTION

Reference will now be made to the accompanying drawings, which at least assist in illustrating the various pertinent features of the present invention. The description is not intended to limit the invention to the form disclosed herein. Consequently, variations and modifications commensurate with the following teachings, and skill and knowledge of the relevant art, are within the scope of the present invention. The embodiments described herein are further intended to explain the best modes known of practicing the invention and to enable others skilled in the art to utilize the invention in such, or other embodiments and with various modifications required by the particular application(s) or use(s) of the present invention.

### Exemplary Implantable System

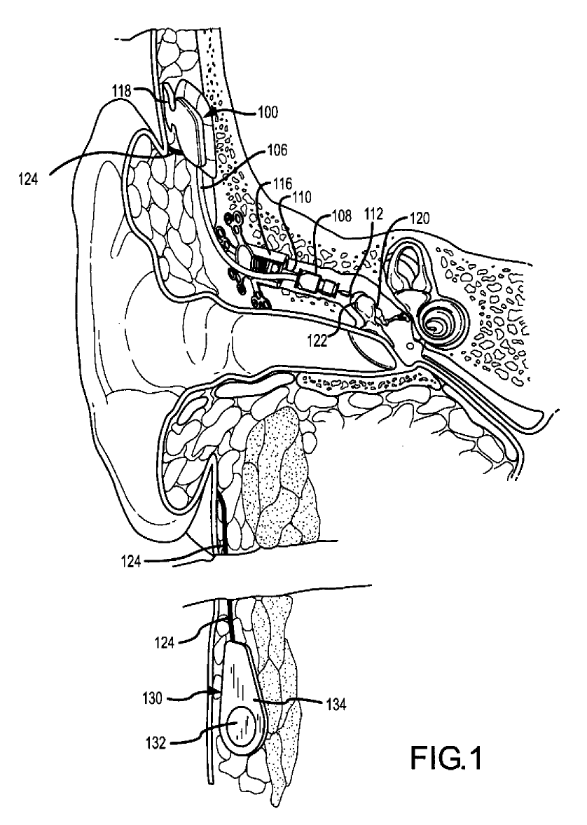

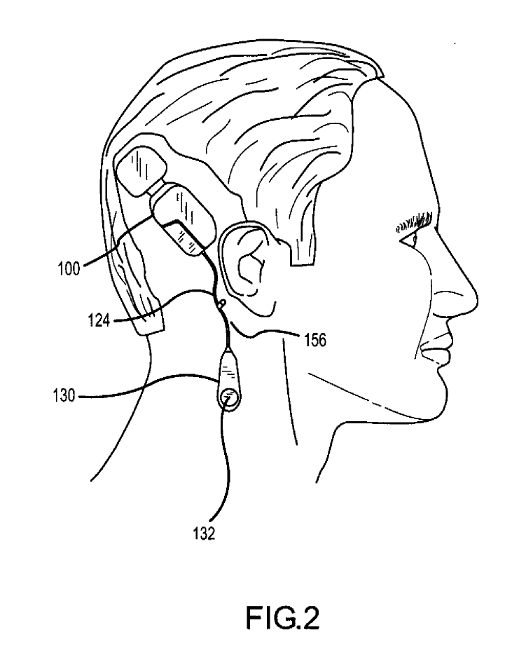

FIGS. 1 and 2 illustrate one application of the present invention. As illustrated, the application comprises a fully implantable hearing instrument system. As will be appreciated, certain aspects of the present invention may be employed in conjunction with semi-implantable hearing instruments as well as fully implantable hearing instruments. Therefore the illustrated application is presented for purposes of illustration and not by way of limitation.

In the illustrated system, a biocompatible implant housing 100 is located subcutaneously on a patient's skull. The implant housing 100 includes a signal receiver 118 (e.g., comprising a coil element) and is interconnected to a microphone assembly 130 via a signal wire 124. The implant housing 100 may be utilized to house a number of components of the implantable hearing instrument. For instance, the implant housing 100 may house an energy storage device and a signal processor. Various additional processing logic and/or circuitry components may also be included in the implant housing 100 as a matter of design choice. In the present arrangement, the signal processor within the implant housing 100 is electrically interconnected via a signal wire 106 to a transducer 108.

The transducer 108 is supportably connected to a positioning system 110, which in turn, is connected to a bone anchor 116 mounted within the patient's mastoid process (e.g., via a hole drilled through the skull). The transducer 108 includes a connection apparatus 112 for connecting the transducer 108 to the ossicles 120 of the patient. In a connected state, the connection apparatus 112 provides a communication path for acoustic stimulation of the ossicles 120, e.g., through transmission of vibrations to the incus 122. To power the fully implantable hearing instrument system of FIG. 1, an external charger (not shown) may be utilized to transcutaneously re-charge an energy storage device within the implant housing 100.

The microphone assembly 130 is separate and spaced from the implant housing 100 such that it is not mounted to the skull of a patient. The microphone assembly 130 includes a diaphragm 132 that is positioned to receive ambient acoustic signals through overlying tissue, a microphone transducer (not shown) for generating an output signal indicative of the received ambient acoustic signals, and a housing 134 for supporting the diaphragm 132 relative to the transducer. As shown, the microphone assembly 130 is mounted to soft tissue of the neck of the patient and the wire 124 interconnecting the implant housing 100 and the microphone assembly 130 is routed subcutaneously behind the ear of the patient.

During normal operation, acoustic signals are received subcutaneously at the diaphragm 132 of the microphone assembly 130. The microphone assembly 130 generates an output signal that is indicative of the received acoustic signals. The output signal is provided to the implant housing 100 via a signal wire 124. Upon receipt of the output signal, a signal processor within the implant housing 100 processes the signals to provide a processed audio drive signal via a signal wire 106 to the transducer 108. The audio drive signal causes the transducer 108 to transmit vibrations at acoustic frequencies to the connection apparatus 112 to effect the desired sound sensation via mechanical stimulation of the incus 122 of the patient.

As noted above, the microphone assembly 130 may be spaced from the implant housing 100 such that it need not be mounted on the skull of a patient. By spacing the microphone assembly 130 away from the skull, vibrations within the skull that may result from, for example, transducer feedback and/or biological sources (e.g., talking and/or chewing) may be attenuated prior to reaching the microphone assembly 130. Stated otherwise, mounting the microphone assembly 130 relative to soft tissue of the patient may isolate the microphone assembly 130 from one or more sources of non-ambient vibrations (e.g., skull-borne vibrations).

As shown in FIG. 2, the microphone assembly 130 may be mounted in the soft tissue of a patient's neck. In the present embodiment, the microphone assembly may be positioned below the tip 156 of the mastoid process on the patient's skull. Positioning the microphone assembly 130 at a position proximate to the mastoid tip allows implantation of the microphone assembly 130 through an incision formed for the hearing instrument. That is, a surgeon may tunnel down from the hearing instrument incision and form a small pocket for the microphone assembly 130 beneath the skin of the patient's neck. Accordingly, the wire 124 interconnecting the implant housing 100 and the microphone assembly 130 may be routed during such a procedure. However, it will be appreciated that other soft tissue placements are possible and within the scope of the present invention.

In any soft tissue placement, patient tissue may be disposed between any underlying bone and the microphone assembly 130. That is, the microphone assembly may be not in direct contact with a bone surface as such surfaces are highly effective in transferring vibrations to the microphone assembly. It may be desirable that at least 2 mm of soft tissue be disposed between the microphone assembly and any underlying bone. In order to maintain the position of the assembly 130 relative to the soft tissue, the assembly may be appropriately sutured to such soft tissue. While the soft tissue mount allows for attenuating and/or substantially eliminating the transfer of skull borne vibrations/noise to the microphone assembly 130, it may still be desirable to process the microphone output signal(s) to reduce the effect of such noise. One arrangement that may be utilized to reduce the effects of non-ambient sound is described in U.S. patent application Ser. No. 11/330,788 entitled: “Active vibration attenuation for implantable microphone,” having a filing date of Jan. 11, 2006, the entire contents of which are incorporated herein by reference.

While removal of an implanted microphone from the surface of a patient's skull may provide for attenuation of some forms of biological noise, such microphone removal may raise additional issues. Specifically, while it may be possible to move one or more components of an implantable hearing system to a soft tissue location to eliminate the need, for example, of forming a bone bed for that component, such soft tissue mounted implantable components can migrate subcutaneously. That is, as opposed to implantable instruments that are securely affixed to an underlying bone, soft tissue mounted components may have some limited movement subcutaneously.

Furthermore, a portion of the population that utilizes implantable devices has a tendency to manually manipulate these devices transcutaneously. That is, a number of implant wearers are considered “twiddlers” who have a tendency to consciously or subconsciously feel and/or apply forces to implanted devices. Accordingly, when such devices are mounted in soft tissue, such twiddling may result in damage to the device and/or to tissue surrounding the implantable device. Accordingly, methods and devices are provided herein that allow for improved interconnection between an implantable component and soft tissue. These methods and devices reduce subcutaneous migration of implanted components and/or reduce the stresses that may be applied to such components due to such migration.

### Anti-Migration

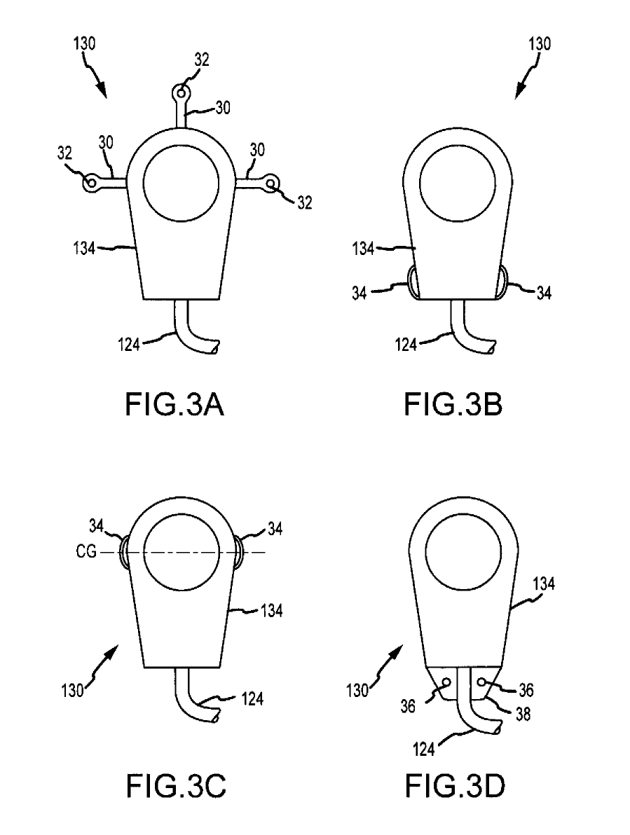

The systems and methods discussed herein are primarily directed to enhancing the interconnection between an implanted microphone and surrounding soft tissue. However, it will be appreciated that such systems and methods are applicable to other implantable devices. FIGS. 3A-3D illustrate four embodiments of an implantable microphone assembly 130 including one or more retention elements, or securement members, for use in securing the assembly 130 to soft tissue. As illustrated in FIG. 3A, the retention elements or securement members may be formed of any appropriate number of legs, wings, arms or loops (e.g., three) that are interconnected to a periphery of the assembly 130 and that radiate axially from the center of the assembly 130. These legs 30 may each include one or more apertures 32 on their distal end. Accordingly, when the microphone assembly is positioned subcutaneously, the legs may extend over underlying tissue, and one or more soft tissue securement devices (e.g., tissue sutures, not shown) may be placed through each of the apertures 32 to secure the assembly relative to the underlying soft tissue. As the legs 30 may extend axially in different directions from the housing 134 of the assembly 130, the overall width of the assembly 130 may be greatly increased. In this regard, a microphone assembly utilizing such legs may have an increased width which may make the assembly more difficult for a person to manipulate transcutaneously. That is, it may be more difficult to turn or roll the assembly.

FIG. 3B illustrates a microphone assembly that utilizes first and second securement members in the form of suture retention loops 34. In the present embodiment, the suture retention loops 34 may be interconnected to the housing 134 of the microphone assembly 130 near where the signal wire 124 connects to the housing 134. Such suture retention loops 34 may be interconnected to the housing 134 in any appropriate manner including, without limitation, via welding and soldering. Alternatively, such retention loops 34 may be integrally formed with the housing 134. In this embodiment, soft tissue securement devices (e.g., sutures) may be placed through the suture loops 34 to secure the assembly 130 to underlying tissue.

FIG. 3C illustrates another embodiment that utilizes first and second suture loops 34 that are attached to the microphone housing 134. In this embodiment, the suture loops 34 may be attached to the housing 134 along the center of gravity of the microphone housing. Such connection above the center of gravity of the housing 134 may reduce the relative movement of the assembly to overlying tissue. By attaching the microphone assembly to soft tissue about the center of gravity, the housing 134 may move with the surrounding tissue. For instance, a user's own voice may produce a tissue pressure wave that passes through the soft tissue in which the microphone assembly is mounted. By being mounted about its center of gravity the microphone assembly is permitted to move in unison with the tissue pressure wave. This reduces the relative movement between the microphone diaphragm and overlying tissue due to the tissue pressure wave. Accordingly, this reduces noise in the microphone output caused by such relative movement.

FIG. 3D illustrates another embodiment of a microphone assembly 130 that utilizes one or more suture apertures. In the present embodiment, first and second suture apertures 36 are formed within a strain relief element 38 disposed on one end of the housing 134. In such an arrangement, the strain relief element 38 may provide strain relief for the signal wire 124 where it enters into the microphone housing 134. Such an arrangement may reduce the amount of force applied to the signal wire 124 by the housing 134 while still providing securement to underlying patient tissue.

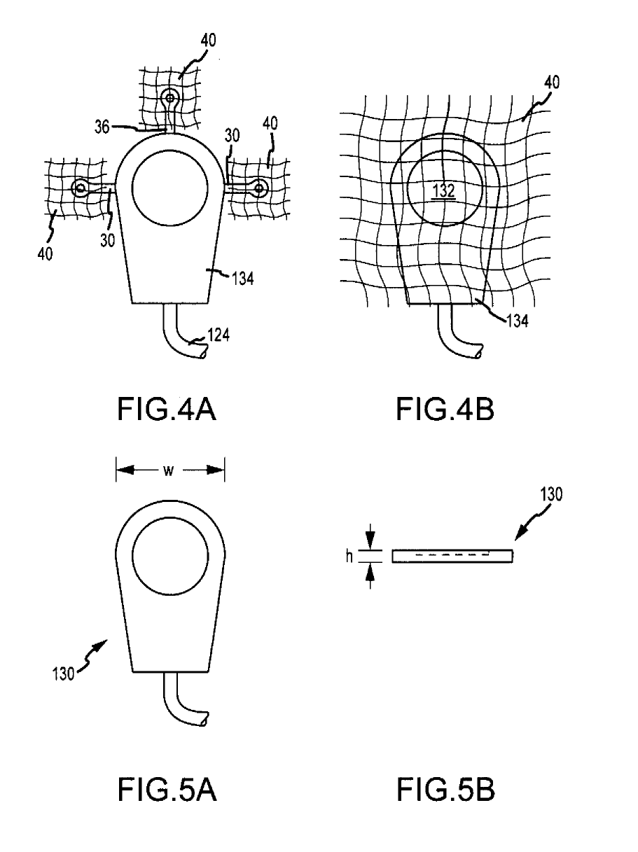

FIGS. 4A and 4B illustrate use of a mesh member (e.g., mesh fabric) to reduce relative movement of an implantable component such as a microphone assembly relative to patient tissue. As shown in FIG. 4A, one or more securement members in the form of radial legs or wings 30 extend from the housing 134. Covering the distal ends of each of these legs/wings is a mesh fabric 40. In such an arrangement, the mesh fabric may be formed as a pocket into which the distal end of a leg 30 is received. One exemplary mesh fabric that may be utilized is a PTFE mesh such as Gore-Tex®. However, it will be appreciated that other fabrics may be utilized as well. What is important is that the mesh fabric 40 allows for tissue ingress or growth once implanted. That is, during the healing process, tissue may grow into and/or through apertures of the mesh of the fabric thereby providing an enhanced interconnection between the fabric and the tissue. Accordingly, if the legs/wings 30 are securely interconnected to the mesh (e.g., sutured through the aperture 32), the mesh fabric may provide secure interconnection between the housing and surrounding tissue. Accordingly, such mesh fabric 40 may significantly reduce the potential for migration of the implanted housing subcutaneously.

FIG. 4B illustrates a further embodiment of use of a mesh fabric 40 to limit the migration of the implantable housing 134. In this embodiment, the mesh fabric 40 may appropriately cover, or encapsulate, a portion of the housing 134. For instance, the mesh fabric 40 may encapsulate an entirety of the housing 134. Again, the mesh fabric 40 may define a pocket or sleeve into which the housing 134 may be disposed. Further, it will be appreciated that an aperture may be made in the mesh fabric that is sized and positioned above the diaphragm 132 of the microphone assembly 130 (not shown) to enhance the functionality of the diaphragm 132. Once tissue ingresses into the mesh fabric 40 a secure attachment may be formed between the tissue and the housing 134.

FIGS. 5A and 5B illustrate another embodiment of an implantable housing that is resistant to subcutaneous migration. In the embodiment of FIG. 5A and 5B, the microphone housing 134 has a very high aspect ratio. That is, the cross-sectional width w of the microphone is at least 2.5 times larger than the height h of the microphone. It will be appreciated that this may reduce the protuberance of the microphone through the skin of a wearer as well as reduce the ability of a wearer to overturn such an implanted housing. That is, once tissue heals around the edges of the high aspect ratio/low profile housing, the large difference in the width-to-height prevents the assembly from being overturned.

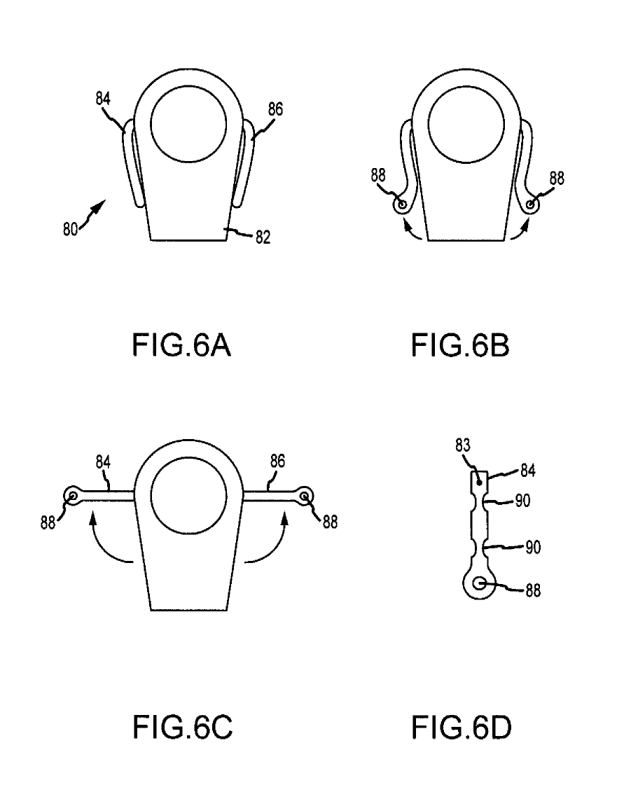

FIGS. 6A-6D illustrate a further embodiment of an implantable microphone assembly 80 that may be utilized for subcutaneous positioning. More particularly, the embodiment of FIGS. 6A-D illustrates a microphone assembly that may be utilized for positioning relative to both soft tissue and underlying bone. That is, in a first configuration, the microphone assembly 130 of FIGS. 1-2 may be adapted for interconnection to soft tissue and in a second configuration may be adapted to connection to underlying bone. As shown in FIG. 6A, the microphone assembly 80 has one or more securement members such as first and second mounting legs 84, 86 attached to the microphone housing 82. These mounting legs 84, 86 may be designed to bend in response to an applied force. Initially, the legs 84, 86 may be disposed in near conformance with the side edges of the microphone housing 82. Depending on the application, the legs 84, 86 may be bent to provide an appropriate connecting mechanism.

The microphone assembly 80 may allow for soft tissue placement with or without sutures. That is, if no sutures are desired, the first and second legs 84, 86 may be left in an undeformed state substantially aligned with the outside surfaces of the microphone housing 82. In such an arrangement, it may be desirable to associate one or more mesh members with one or more various portions of the housing 82 or securement members. Alternatively, if sutures are desired to maintain the subcutaneous location of the microphone assembly 80, the legs 84, 86 may be deformed to an extent such that they lie adjacent to soft tissue structures suitable for suturing. As illustrated in FIG. 6b, the ends of the legs 84 may be bent outwards to expose one or more suture apertures 88 that are disposed near the microphone housing 82. In instances where it is desirable to interconnect the microphone assembly 80 to underlying bone, one or more of the first and second legs 84, 86 may be extended outward from the sides of the microphone housing 82 as shown in FIG. 6C. In such an arrangement, the legs 84, 86 may be deformed to match the contour of an underlying bone surface. In this case, the apertures 88 may be utilized to receive a bone screw to interconnect the microphone assembly 80 to such underlying bone. It will be appreciated that movement of the first and second legs 84, 86 from a stowed position along the sides of a housing 82 as shown in FIG. 6A to the extended position as shown in FIG. 6C may further entail rotating the ends of the legs 84, 86 to properly orient the suture apertures 88 with an underlying structure.

FIG. 6D illustrates one arm 84 that may be utilized with the embodiment of FIG. 6A-6C. As shown, the arm 84 may include a hole 83 (e.g., laser spot) that may be utilized for spot welding the leg 84 to the housing 82. However, it will be appreciated that other attachment mechanisms may be utilized. As shown, the arm 84 may include at least one flexible portion 90. In particular, the flexible portion 90 may be disposed between the end of the leg that attaches to the housing and the distal end of the leg including the aperture 88. The flexible portion 90 may have a cross-sectional dimension smaller than that of the adjacent portions of the leg 84. Accordingly, the flexible portion(s) 90 has a bending resistance that is less than that of the bending resistance of the adjacent portions of the leg. Of note, this reduced cross-sectional diameter may further incorporate a different cross-sectional shape (e.g., round vs. rectangular). What is important is that the flexible portion 90 will, upon application of an applied stress, deflect or bend prior to another portion of the mounting leg bending. Accordingly, this may facilitate the extension of the legs 84, 86 and/or the conformance of such legs 84, 86 relative to underlying structure.

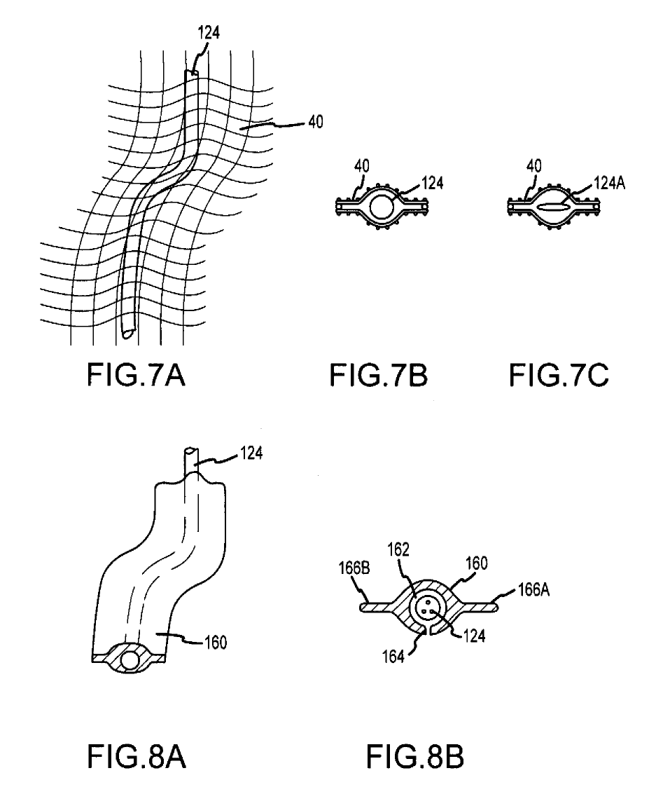

In addition to the desirability of limiting the migration of the implantable microphone assembly or other implantable housings, it may also be desirable to limit the migration of a signal wire extending between two such implantable components. For instance, referring to FIG. 2, it is noted that the signal wire 124 may extend between the implant housing 100 and the microphone assembly 130. Like the soft tissue mounted microphone assembly 130, the signal wire 124 may be subject to external manipulation. FIGS. 7A and 7B illustrate one embodiment of a system for use in limiting the subcutaneous migration of a signal wire. As shown, the signal wire 124 may be appropriately covered by, or encased within, a mesh member in the form of a fabric mesh 40 similar to that discussed above in relation to FIGS. 4A and 4B. In such an arrangement, the fabric mesh 40 may be designed as a sleeve that fits over at least a portion of the length of the signal wire 124. Again, the fabric mesh 40 may have an open structure (e.g., includes a number of apertures) that allows for tissue ingress during the healing process. Accordingly, once the tissue is ingressed into the fabric mesh 40, subcutaneous movement/migration of the signal wire 124 is significantly limited. In a further arrangement, shown in FIG. 7C, a low profile signal wire 124a is utilized. As shown, the low profile signal wire is substantially ovular in shape. In this regard, when disposed beneath the skin the profile beneath the skin is reduced. Accordingly, use of such a low profile signal wire may reduce the tendency for people to manipulate the wire 124a consciously and/or subconsciously. The wire 124a may also be appropriately covered by, or encased within the fabric mesh 40.

### Strain Relief

In addition to limiting the migration of subcutaneously implantable components, it may also be desirable to reduce the strain applied to one or more signal wires connecting these components. As will be appreciated, if a first implantable component is affixed to the patient's skull (e.g., an implantable signal processor) and a second component is fixed to soft tissue within a patient's neck (e.g., microphone), the distance between these components may change slightly based on the posture of an individual. Specifically, if an individual turns their head, the distance between these components may increase or decrease. Accordingly, there may be a strain or other force applied to a signal wire connecting such components.

In order to alleviate the strain applied to a signal wire connecting implanted components, it is typically desirable to route the signal wire with some slack (e.g., a relief bend such as an S-bend). Accordingly, if a wearer of the device increases the distance between the components, the relief bend may allow for lengthening the wire and accommodating the turn of the patient's head.

FIGS. 8A and 8B illustrate one embodiment of a strain relief device 160 that may be attached to a subcutaneous signal wire 124. As shown, the strain relief device may have an S-bend or jog along its length. Of further note, the strain relief device 160 may be formed of a resilient material such as, for example, silicone elastomer. While being resilient, the strain relief device may have a preformed S-bend shape that maintains slack between the ends of the signal wire 124. Accordingly, when opposing ends of the signal line are pulled, the resilient strain element 160 may slightly straighten to accommodate a change in length of the signal wire 124.

In the present embodiment, the strain relief device 160 may be formed to snap onto the signal wire 124. In this regard, the strain relief device may include a central lumen 162 that extends through the length of the device 160. Accordingly, the signal wire 124 may be disposed through this lumen. In the present embodiment, the central lumen 162 may include an access slot 164 through which the signal wire 124 may be disposed. It will be further appreciated that the strain relief device 160 may include one or more apertures within opposing surfaces 166a, 166b that may be utilized to secure (e.g., suture) the strain relief element to underlying tissue and/or anchors the strain relief element to underlying bone.

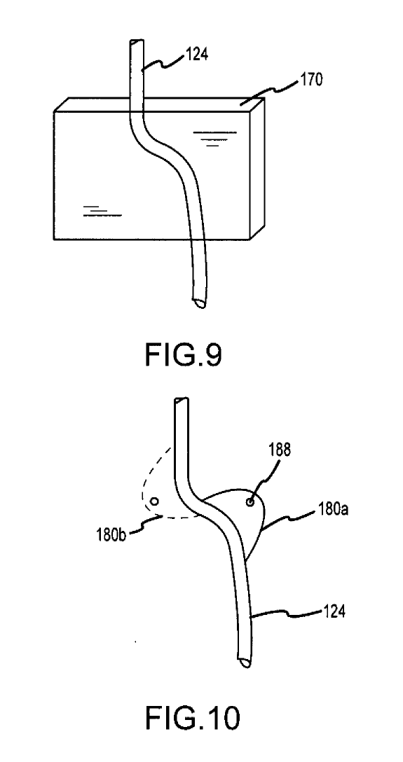

FIG. 9 illustrates a second embodiment of a strain relief element 170. In this embodiment, the strain relief element 170 may be formed of a resilient block molded over the signal wire 124. In such an arrangement, a silicon material may be formed over the signal wire 124 while the signal wire has a desired strain relief shape (e.g., S-bend).

FIG. 10 illustrates a further embodiment of a strain relief element 180 that may be attached to a signal wire 124. In this embodiment, the strain relief element 180 may be formed of a resilient anchor 180a interconnected to one or more locations along the length of the signal wire 124. This resilient anchor 180a may include an aperture 188 that may be anchored to underlying tissue and/or bone. Utilization of two such anchors 180a, 180b may allow for resiliently interconnecting the signal wire to two locations to form a desired strain relief configuration for the signal wire 124.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A system for subcutaneous securement of an implantable housing to soft tissue, comprising:
at least one securement member that is at least one of interconnected to and disposable over at least a portion of the housing, wherein the at least one securement member at least one of extends away from and is selectively extendable away from a periphery of the housing; and
at least one aperture extending through the at least one securement member, wherein the at least one aperture is adapted for selective receipt of one of a soft tissue securement device and soft tissue growth therethrough.

2. The system of claim 1, wherein the at least one securement member comprises a plurality of securement members spaced about and interconnected to the periphery of the housing, wherein the plurality of securement members extend away from the housing in a corresponding plurality of different directions, and wherein each of the plurality of securement members includes an aperture therethrough that is adapted for selective receipt of a soft tissue securement device therethrough.

3. The system of claim 2, wherein each of the plurality of securement members comprises at least one of a leg, arm, wing and loop.

4. The system of claim 2, further comprising:
a plurality of soft tissue securement devices each in the form of a tissue suture, wherein different ones of said tissue sutures are selectively receivable through different ones of the apertures of the plurality of securement members and soft tissue.

5. The system of claim 4, further comprising:
at least one mesh member selectively positionable over at least one of the plurality of securement members and at least one corresponding tissue suture.

6. The system of claim 5, wherein the mesh member is sized for growth of soft tissue through the mesh member.

7. The system of claim 5, wherein the mesh member comprises a pocket that is sized for selective receipt of at least one securement member of the plurality of securement members.

8. The system of claim 2, wherein at least two securement members of the plurality of securement members extend along an axis that intersects the center of gravity of the housing.

9. The system of claim 1, wherein the at least one securement member comprises a mesh member that is selectively positionable over a portion of the housing.

10. The system of claim 9, further comprising at least one soft tissue securement device that is selectively receivable through at least one aperture of the mesh member and soft tissue.

11. The system of claim 9, wherein the mesh member comprises a pocket that is sized for selective receipt of the housing.

12. The system of claim 1, further comprising:
a signal wire interconnected to the housing.

13. The system of claim 12, further comprising:
a strain relief member positioned about at least a portion of a length of the signal wire.

14. The system of claim 12, further comprising a mesh member that is disposable over at least a portion of the signal wire.

15. A method for use with an implantable housing, comprising:
providing at least one securement member that is at least one of interconnected to and disposable over at least a portion of an implantable housing, wherein the at least one securement member at least one of extends away from and is selectively extendable away from a periphery of the implantable housing, wherein the at least one securement member comprises at least one aperture that extends through the at least one securement member, and wherein the at least one aperture is adapted for selective receipt of one of a soft tissue securement device and soft tissue growth therethrough;
positioning the implantable housing at a subcutaneous location, wherein the implantable housing is supported by soft tissue and is spaced from a surface of the skull of a patient; and
utilizing the at least one aperture of the at least one securement member to secure the implantable housing to the soft tissue.

16. The method of claim 15, wherein the utilizing step comprises disposing a soft tissue securement device through at least one securement member that is interconnected to a portion of the implantable housing.

17. The method of claim 16, wherein the disposing step comprises extending the soft tissue securement device in the form of a tissue suture through the at least one aperture of the at least one securement member and soft tissue.

18. The method of claim 17, wherein the providing step comprises providing a plurality of securement members spaced about and interconnected to a periphery of the implantable housing, wherein the plurality of securement members extend away from the implantable housing in a corresponding plurality of different directions, wherein each of the plurality of securement members includes an aperture therethrough that is adapted for selective receipt of a soft tissue securement device therethrough, and wherein the disposing step comprises extending different ones of a plurality of tissue securement devices in the form of a number of tissue sutures through different ones of the apertures of the plurality of securement members and soft tissue.

19. The method of claim 17, further comprising:
locating a mesh member over the at least one securement member; and
extending the tissue suture through the mesh member.

20. The method of claim 19, wherein the locating step comprises inserting the at least one securement member into a pocket formed in the mesh member.

21-31. (canceled)
